(19) United States
(12) Patent Application Publication
(10) Pub. No.: US 2011/0065914 A1
(43) Pub. Date: Mar. 17, 2011

(54) PERFLUORO MACROCYCLES IN 18F-LABELLING OF MACROMOLECULES

(76) Inventors: BENGT Langstrom, Uppsala (SE); Johan Ulin, Uppsala (SE); Farhad Karimi, Massachusetts (SE)

(21) Appl. No.: 12/673,853

(22) PCT Filed: Aug. 28, 2008
(86) PCT No.: PCT/US08/74558

§ 371 Date: Nov. 30, 2010

Related U.S. Application Data
(60) Provisional application No. 60/968,692, filed on Aug. 29, 2007.
(60) Provisional application No. 60/969,012, filed on Aug. 30, 2007.

(51) Int. Cl. C07D 498/08

(52) U.S. Cl. 540/469

(57) ABSTRACT

The invention relates to a synthetic strategy of using perfluoro crown ethers and other macrocycles to bind to [18F]-labelled fluorination reactions. The optional use of implementing perfluoro kryptofix 2.2.2 in this process is also disclosed. The present invention also claims perfluoro kryptofix structures that are suitable for use in 18F-labelling of fluorous based structures.

### FIELD OF THE INVENTION

The invention relates to a synthetic strategy of using perfluoro crown ethers and other macrocycles to bind to [¹⁸F]-labelled fluorination reactions. The optional use of implementing perfluoro kryptofix 2.2.2 in this process to simplifying the [¹⁸F]-labelled fluorination reactions and thus obtaining a faster reaction time is also disclosed. The present invention claims perfluoro kryptofix structures that are suitable for using in 18F-labelling of fluorous based structures.

### BACKGROUND

In general, fluorine is a small atom with a very high electronegativity. Covalently bound fluorine is larger than a hydrogen atom but occupying a smaller van der Waal's volume than a methyl, amino or hydroxyl group. Fluorine substituent effects on pharmacokinetics and pharmacodynamics are very obvious. Eckelman W C. Nucl Med Bio 2002; 29: 777-782. Therefore, the replacement of a hydrogen atom or a hydroxy group by a fluorine atom is a strategy frequently applied in both positron emission tomography (“PET”) tracer and drug developments. Id. The replacement of a hydrogen atom by a fluorine atom can alter the pKa, the dipole moments, lipophilicity, hydrogen bonding, the chemical reactivity, the oxidative stability, the chemical reactivity of neighboring groups or metabolic processes. Smart B. E. J Fluorine Chemistry 2001; 109: 3-11. The replacement of a hydroxyl group is based on the hypothesis that fluorine is a hydrogen acceptor like the oxygen of a hydroxyl group. Czermin J and Phelps M. Annu Rev Med 2002; 53: 89-112.

The increasing use of PET for clinical diagnosis, drug development, and more generally, biological research has prompted many chemists to develop new labelling or purification methods. Amongst the commonly used positron-emitting isotopes, ¹⁸F stands out because of its advantageous half-life of 110 minutes. In addition, the low positron energy of ¹⁸F results in the formation of images of high resolution. Furthermore, for most types of reactions introduced in utilizing PET, electrophilic fluorinations offer exciting opportunities to access ¹⁸F-labelled compounds otherwise unreachable or difficult to prepare via nucleophilic fluorination.

Fluorine-18 as used in PET has excellent nuclear properties such as low positron energy that results in low radiation dose, short maximum range in tissue and convenient half-life (t_{1/2}=110 min) considering distribution to other hospitals and performing longer acquisition protocols.

Furthermore, the application of radiolabelled bioactive peptides for diagnostic imaging is gaining importance in nuclear medicine. Biologically active molecules, which selectively interact with specific cell types, are useful for the delivery of radioactivity to target tissues. For example, radiolabelled peptides have significant potential for the delivery of radionuclides to tumours, infarcts, and infected tissues for diagnostic imaging and radiotherapy. ¹⁸F is the positron-emitting nuclide of choice for many receptor-imaging studies. Therefore, ¹⁸F-labelled bioactive peptides have great clinical potential because of their utility in PET to quantitatively detect and characterise a wide variety of diseases.

Radiolabeling of compounds with [¹⁸F]-fluoride can be achieved either by indirect displacement using fluoroalkylation agents or direct displacement of a leaving group. Using fluoroalkylation agents or direct displacement is not always convenient for all pharmaceutical substrates due to the formation of by-products, low yield, and the difficulties in purification processes. The development of fluorous chemistry also known as ponytail chemistry, (“PT”) in n.c.a. nucleophilic ¹⁸F-fluorination has also shown promising results. Using PT chemistry offers simplifications of the overall process going from [¹⁸F]-fluoride in target water to pure radiopharmaceutical since in the compounds containing the ponytail can easily be removed by SPE-purification where the SPE-matrix contains a ponytail matrix and would then be applied as an alternative to solid phase or surface based chemistry.

Additionally, since the half-life of ¹⁸F is only 110 minutes, ¹⁸F-labelled tracers for PET therefore have to be synthesized and purified as rapidly as possibly, and ideally within one hour of clinical use. Accordingly, it is important to find a chemical compound that would aid in speeding the reaction time and simplify the purification of ¹⁸F-fluorination reactions.

Kryptofix 2.2.2 (also known as 4,7,13,16,21,24 hexaoxa-1,10-diazabicyclo[8,8,8] hexacosane) is a toxic compound which has been used to improve ¹⁸F-fluorination reactions. However, there is a need to simplify purification and speed the reaction time for ¹⁸F-fluorination reactions that pertain to PET imaging. Specifically, the present invention demonstrates that applying perfluoro kryptofix to ¹⁸F-fluorination reactions will both simplify and speed up the reaction time.

Furthermore, antisense oligonucleotides (“ODN”) play an important role in ¹⁸F-labelled tracers for PET. ODN's are typically 10-25 nucleotides long, single-stranded DNA or RNA molecules that can bind to their complementary DNA or RNA sequence via Watson-Crick base pairing. When the sequence of the target gene is known, antisense ODNs consisting of at least 15-17 nucleotides can be designed that are capable of selective hybridization to a single gene of interest within the entire human genome. Hybridization of an antisense ODN to its target mRNA prevents the translation process to occur and thus the undesired expression of a specific gene can be inhibited. The exquisite specificity of base pair formation has initiated great interest in ODNs not only as potential therapeutics Lancet, 2001, vol. 358, pgs. 489-497, but also as diagnostic agents J. Nucl. Med. 1999, vol. 40, pgs. 693-703. Non-invasive imaging of the hybridization of ODNs to their target mRNA would enable the selective detection of the expression of specific genes. In addition, imaging of radiolabeled ODNs could provide the means to study the distribution and pharmacokinetics of ODNs in living subjects.

PET (PET/CT) is sensitive and non-invasive with the unique capability to quantitatively measure pharmacological, biochemical and metabolic processes in vivo. Thus, PET could be a versatile tool to assess in vivo behavior of radiolabeled ODNs Curr. Pharm. Des. 2002, vol. 8, pgs. 1451-1466. Kobori and coworkers recently labelled antisense phosphorothioate ODNs with the positron emitter carbon-11 for PET imaging Neuroreport, 1999, vol. 10, pgs. 2971-2974. The half-life of ¹¹C (20 min); however, is rather short for antisense ODN imaging, because cellular uptake and efflux of ODNs are relatively slow processes J. Nucl. Med. 2001, vol. 42, pgs. 1660-1669. In this respect, fluorine-18 has a more favorable half-life of 110 min. Accordingly, the present Inventors have used 4-([¹⁸F]-fluoromethyl)phenyl isothiocyanate to label ODNs modified with a hexylamine moiety at the 5′-terminus Acta Chem. Scand. 1997, vol. 51, pgs. 1236-1240, but they observed loss of the fluorine-18 label, due to solvolysis. The present Inventors also applied the chemically more stable precursor N-succinimyl 4-[¹⁸F]-fluorobenzoate Acta Chem. Scand. 1998, vol. 52, pgs. 1034-1039 and N-succinimidyl 4-[⁷⁶Br] 76Br-bromobenzoate Acta Chem. Scand. 1999, vol. 53, pgs. 508-512 to label hexylamine-modified ODNs with ¹⁸F and ⁷⁶Br, respectively, but the isolated radiochemical yields remained moderate (7-25%). Another approach was published by the Orsay group, who labeled ODNs with a thiophosphate moiety at the 3′-terminus and various modifications of the sugar phosphate backbone J. Label. Compd. Radiopharm. 1997, vol. 39, pgs. 319-330; J. Label. Compd. Radiopharm. 2000, vol. 43, pgs. 837-848. Conjugation of these modified ODNs at the 3′-terminus with N-(4-[¹⁸F]-fluorobenzyl)-2-bromoacetamide afforded ¹⁸F-labeled ODNs in high radiochemical yield (70-90%). However, a major drawback of this strategy is the rather laborious and lengthy labelling procedure (3-3.5 hours).

This above-mentioned work was further extended into the use of ⁶⁸Ga instead of ¹⁸F. Specifically, as it relates to the present invention, ⁶⁸Ga has been used as a metallic cation for complexation reactions with chelators, naked or conjugated, with peptides or other macro-molecules Bioconjugate Chem. 2004, vol. 15, pgs. 554-560. There is still a need for using perfluoro macrocycles such as perfluorocryptand [2.2.2] as a chelator, that is naked or conjugated to macromolecules for use in ¹⁸F-fluorination. The present invention sets forth a method to solve this need.

Discussion or citation of a reference herein shall not be construed as an admission that such reference is prior art to the present invention.

### SUMMARY OF THE INVENTION

There is a need for using naked or conjugated chelators that are attached to macromolecules in ¹⁸F-fluorination reactions since these chelators would improve the labelling procedure and radiochemical yield of these reactions greatly. A naked chelator is defined as a binding to a structure that is too tiny to see by the naked eye (less than 1 millimeter). A conjugated chelator are all chelators defined outside the scope of the naked chelator.

It is also important to note that the use of Kryptofix 2.2.2 (also known as 4,7,13,16,21,24 hexaoxa-1,10-diazabicyclo[8,8,8] hexacosane) with perfluoro structural attachments can further aid in the purification of the final product (upto 5%) as well as reducing the reaction time to obtain the product by one fourth of the time.

### DETAILED DESCRIPTION OF THE INVENTION

The use of perfluoro chemistry is advantageous in improving ¹⁸F-fluorination reactions. Specifically, using Kryptofix 2.2.2 (also known as 4,7,13,16,21,24 hexaoxa-1,10-diazabicyclo[8,8,8] hexacosane) with perfluoro structural attachments aid in the purification of the final product as well as reducing the reaction time to obtain the product by one fourth of the time. Also the use of perfluoro Kryptofix 2.2.2 is advantageous if separating kryptofix is difficult.

Furthermore, the presence of Kryptofix 2.2.2 has a detrimental effect on the fluoridation of iodonium salts—presumed to be via the formation of a radical alpha to the Kryptofix nitrogens. However, by using a perfluoro attachment to the Kryptofix removes this problem.

Additionally, the perfluoro molecules proposed do allow nucleophilic fluoridation which was not the case when only Kryptofix 2.2.2 was used in ¹⁸F fluorination reactions.

One embodiment of the present invention in making sure the property of Kryptofix 2.2.2 stays together by coupling it with a perfluoro tail. Examples of the perfluoro Kryptofix are as follows:

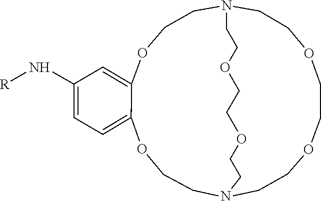

wherein R=R_{f}, COR_{f}, where _{f} can be 1 to 20.

Another possible perfluoro krytofix used would be:

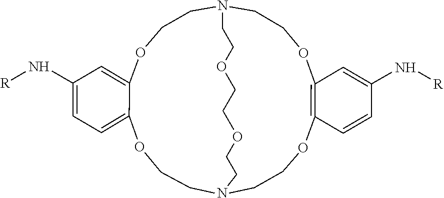

wherein R=R_{f}, COR_{f} where _{f} can be 1 to 20.

It is further important to point out here that perfluoroalkyl sulfonates are not suitable leaving groups for n.c.a. nucleophilic ¹⁸F-fluorination for synthesis of [¹⁸F]fluoromethyl benzene. However, using a pentafluorobenzenesulfonate precursor has shown promising results and thus is a suitable leaving group for ¹⁸F-labeling with moderated reactivity. The ponytail (“PT”) PT-precursor seems to be quite stable for at least 4-6 months. In an attempt to purify the crude ¹⁸F-labeled product using fluoride-solid phase extraction (“F-SPE”), the radio labeled impurities decreased significantly by about 70%. By using a perfluoro Kryptofix 2.2.2 structure in this reaction the purity of the crude 18F-labelled product is improved by 20% thus reducing the radiolabelled impurities by about 90%.

Furthermore, studies with several perfluoro crown ethers and with perfluorocryptand [2.2.2] chelator have shown that such macrocycles tenaciously bind with ¹⁸F. Perfluoro crown ethers and cryptands coordinate anions preferentially over cations. These perfluoro crown ethers (PFC) can be used by applying a suitable PFC-macromolecules i.e. PFC-MM, trap the ¹⁸F as shown below.

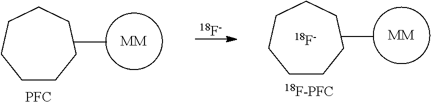

Using this synthetic strategy gives the following advantages:

Half-life of ¹⁸F (110 min) is desirable since cellular uptake and efflux of ODNs are relatively slow processes;

Obtaining a cyclotron produced radionuclide—the labelling might be performed at a PET-site after transferred to the user as ¹⁸F-fluoride; and

This process is an alternative to the potential limitation with labelling of macromolecules using cation ⁶⁸Ga since the half-life is 68 minutes which is half the life of ¹⁸F.

One embodiment of the present invention depicts a process for synthesizing ¹⁸F fluorination reactions according to the following reaction

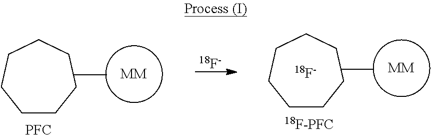

wherein a perfluoro crown ether (PFC) is coupled with a macromolecule (MM) to trap the ¹⁸F- to form ¹⁸F-PFC and a MM.

A further embodiment of the present invention shows that the PFC is perfluorocryptand 2.2.2 or a similar compound thereof and the MM is an amine based group.

Another embodiment of the present invention shows that perfluoro kryptofix 2.2.2 is optionally added to the products.

Yet another embodiment of the invention relates to a radiopharmaceutical kit for the preparation of process (I) and similar structure thereof for use in fluorous PET chemistry.

Still a further embodiment of the invention depicts a method for the use of preparing process (I) and similar structures thereof.

It is further important to point out here that perfluoroalkyl sulfonates are not suitable leaving groups for n.c.a. nucleophilic ¹⁸F-fluorination for synthesis of [¹⁸F]fluoromethyl benzene. However, using a pentafluorobenzenesulfonate precursor has shown promising results and thus is a suitable leaving group for ¹⁸F-labeling with moderated reactivity. The ponytail (“PT”) PT-precursor seems to be quite stable for at least 4-6 months. In an attempt to purify the crude ¹⁸F-labeled product using fluoride-solid phase extraction (“F-SPE”), the radio labeled impurities decreased significantly by about 70%. By using a perfluoro Kryptofix 2.2.2 structure in this reaction the purity of the crude 18F-labelled product is improved by 20% thus reducing the radiolabelled impurities by about 90%.

Still another embodiment of the present invention depicts a compound of the following structure

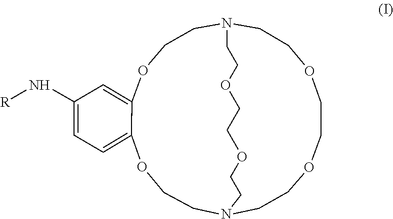

wherein R=R_{f}, COR_{f}, where _{f} can be 1 to 20.

A further embodiment of the present invention depicts a compound of the following structure

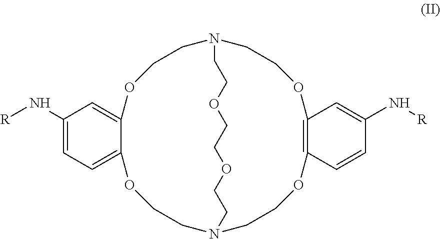

wherein R=R_{f}, COR_{f}, where _{f} can be 1 to 20.

Another embodiment of the present invention depicts a method for the use of preparing compound (I) for use in fluorous PET chemistry wherein the R group of compound (I) is attached to the final product, ¹⁸F-PFC.

A further embodiment of the invention presents a method for the use of preparing compound (II) for use in fluorous PET chemistry wherein the R group of compound (I) is attached to the final product, ¹⁸F-PFC.

The present invention furthermore provides a computer program product for use in carrying out the method and uses of the invention as described herein.

The invention is further described in the following examples, which is in no way intended to limit the scope of the invention.

### Examples of Perfluoro Krytofix 2.2.2

Adding the perfluoro group to Kryptofix 2.2.2 is done by fusing the amine group to the Kryptofix 2.2.2 structure as follows:

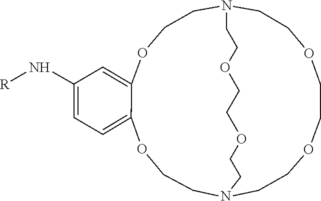

wherein R=R_{f}, COR_{f}. Another possible perfluoro krytofix used would be:

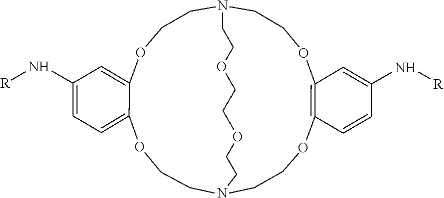

wherein R=R_{f}, COR_{f}.

### Example of a Perfluoro Crown Ether (PFC) to Trap ¹⁸F⁻

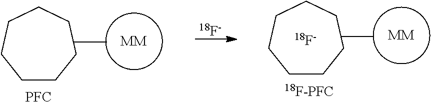

wherein a perfluoro crown ether (PFC) is coupled with a macromolecule (MM) to trap the ¹⁸F- to form ¹⁸F-PFC and a MM.

### Specific Embodiments, Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A process for synthesizing [¹⁸F]-labelled fluorination reactions according to the following reaction: 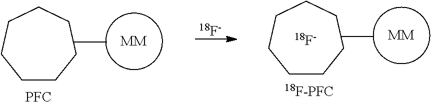
wherein a perfluoro crown ether (PFC) is coupled with a macromolecule (MM) to trap the ¹⁸F- to form ¹⁸F-PFC and a MM.

2. The process according to claim 1, wherein the PFC is perfluorocryptand 2.2.2.

3. The process according to claim 1, wherein the MM is an amine based group.

4. The process according to claim 1, wherein perfluoro kryptofix 2.2.2 is added to the products.

5. A radiopharmaceutical kit for the preparation of claim 1 for use in fluorous PET chemistry.

6. A method for the use of preparing claim 1.

7. A compound of the following structure 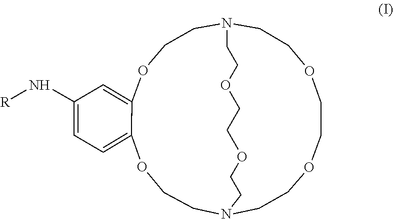
wherein R=R_{f}, COR_{f} where _{f} can be 1 to 20.

8. A compound of the following structure 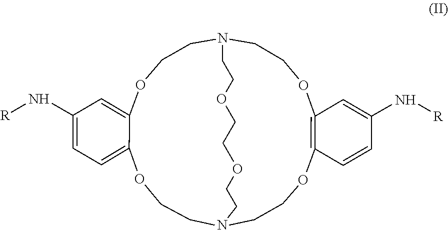
wherein R=R_{f}, COR_{f} where _{f} can be 1 to 20.

9. A method for the use of preparing compound (I) for use in fluorous PET chemistry wherein the R group of compound (I) is attached to the ¹⁸F-PFC of claim 1.

10. A method for the use of preparing compound (II) for use in fluorous PET chemistry wherein the R group of compound (I) is attached to the ¹⁸F-PFC of claim 1.

11. A computer program product for use in carrying out the method of claim 1.
